(19) United States
(12) Patent Application Publication
(10) Pub. No.: US 2002/0031790 A1
(43) Pub. Date: Mar. 14, 2002

(54) Methods for validating polypeptide targets that correlate to cellular phenotypes

(75) Inventor: Carl Alexander Kamb, Salt Lake City, UT (US)

Correspondence Address: MARSHALL, O'TOOLE, GERSTEIN, MURRAY & BORUN, 6300 SEARS TOWER, 233 SOUTH WACKER DRIVE, CHICAGO, IL, 60606-6402, US

(73) Assignee: ARCARIS, INC.

(21) Appl. No.: 09/907,128

(22) Filed: Jul. 17, 2001

Related U.S. Application Data
(63) Continuation of application No. 09/193,759, filed on Nov. 17, 1998, now abandoned.

(51) Int. Cl. G01N 33/567; G01N 33/53; G01N 33/537; G01N 33/543; G06F 19/00; G01N 33/48; G01N 33/50

(52) U.S. Cl. 435/7.21; 435/7.92; 702/19

(57) ABSTRACT

Generally applicable methods for using phenotypic probes to reduce or eliminate false positives, and thereby identify physiologically relevant endogenous target molecules, are provided. The methods use both protein interaction assay steps and phenotypic assay steps. In some embodiments, protein interactions are detected utilizing yeast two hybrid techniques.

### FIELD OF THE INVENTION

The present invention comprises generally applicable methods for identifying endogenous, physiologically relevant cellular components, often endogenous proteins or polypeptides, that are involved in cellular pathways correlating to a phenotype of interest. These cellular components may be readily identified through their interactions with exogenous agents or probes, often “perturbagens,” and are preferably characterized by an ability to bind more than one independent, physiologically relevant perturbagen. By use of these methods, potential therapeutic agents are subjected to parallel validation, and physiologically irrelevant false positives can be readily eliminated.

### BACKGROUND

Most drug development schemes require accurate identification of the endogenous components of physiological pathways that can lead to disease—for example, to cancer. These endogenous components may be potential therapeutic targets, or may point the way to genes that are associated with occurrence of the disease. Identification of such physiologically relevant components (i.e., components that participate in a cellular pathway of interest), however, has been time-consuming and uncertain.

Various protein-protein, protein/DNA, protein/RNA or enzyme-substrate interactions on or within the cell (“endogenous cellular interactions”) may be of particular interest because these interactions provide a means for identifying molecular mechanisms and physiologically relevant components that underlie a disorder or disease state in an organism. For example, once one relevant endogenous cellular interaction is identified, it may be explored in more depth, often enabling the associated physiologically relevant genes and/or cellular pathways to be identified. In addition, a physiologically relevant endogenous cellular interaction provides the basis for screening potential therapeutic agents. It is critical that such endogenous cellular interactions be identified accurately, so that resources are not expended pursuing interactions that ultimately are not physiologically relevant in the target cell.

Using perturbagens to identify relevant endogenous interactions offers advantages in streamlining the identification of physiologically relevant endogenous cellular interactions. Perturbagens often are proteinaceous molecules that interact with endogenous proteins in a cell, and either partially or completely disrupt the normal function of an endogenous cellular pathway. This disruption of specific biochemical interactions generates a correlative “mutant” phenotype, which may in turn be used as a selection characteristic. Perturbagens include proteinaceous moieties (peptides, polypeptides or proteins), nucleic acids, or other compounds.

Even with the advantages of using perturbagens to identify endogenous proteinaceous components, a variety of difficulties inhere in linking any detectable proteinaceous component to the actual physiological pathways in the target cell via specific binding interactions. For example, current systems for detecting protein-ligand or enzyme-substrate interactions often detect false positive results of at least two varieties: (1) interactions that are spurious artifacts of the assay system used to detect the protein-ligand interactions, and which do not reflect bona fide interactions in the endogenous environment of the cell under study (termed herein, “artifactual interactions”), and (2) interactions that do occur in the endogenous cellular environment, but which are not relevant to the cellular pathway of interest (termed herein, “non-relevant interactions”). Conversely, current assay methodologies also provide undesirable false negatives, in which physiologically relevant interactions (interactions relevant to the cellular pathway of interest) evade detection. Moreover, when the sensitivity of an assay is increased so as to decrease false negatives, more false positives may result.

Two general methods are most commonly used to assay for protein-ligand interactions—biochemical methods, and quasi-genetic methods. Both suffer from technical drawbacks.

The biochemical approach is typified by affinity purification techniques that are well known to those of skill in the art. Briefly, affinity purification techniques use a selected protein or peptide as an affinity reagent, which is brought into contact with a reaction mixture. Components that interact with that affinity reagent are then isolated and purified. This general method is of limited utility when the interaction between the target and reagent is not stable or strong, or when proteases that digest one or both of the binding partners are present in the reaction mixture. Moreover, this method undesirably can produce false positives and false negatives, in which a physiologically relevant binding partner that occurs in very low concentrations is not detected due to the presence of more abundant, yet less specifically-bound or strongly interacting proteins. Those proteins are false positives that can compete with the true positive for binding with the affinity reagent and thus mask the presence of the true positive.

The quasi-genetic approach is exemplified by a technique known to those of skill in the art as the two-hybrid assay. E.g., The yeast two-hybrid system, Oxford Univ. Press (1997), Bartel, Paul L. and Fields, Stanley, Ed. This assay often is performed in yeast cells (although it can be adapted for use in mammalian and bacterial cells), and relies upon constructing a first vector having an interaction probe or “bait” that typically is fused to a DNA binding domain (“BD”) moiety, and a second vector having an interaction target or “prey” that typically is fused to a DNA transcriptional moiety (the “activation domain” or “AD”). When the bait and prey interact, the AD and BD moieties are brought into sufficient physical proximity to result in transcription of a reporter gene (e.g., the His3 gene) located downstream of the bound complex. Prey/bait interactions are then detected by identifying yeast cells that are expressing the reporter gene—e.g., which are able to grow in the absence of histidine.

Although the yeast two-hybrid assay system is commonly used to detect protein-ligand interactions, it is known that the assay system produces false positives of several varieties. For example, in some situations the BD fusion moiety of the assay may “self-activate,” thus causing transcription of the downstream reporter gene even though there has not been a prior binding event between the BD-associated bait and the AD-associated prey (one example of an “artifactual interaction”). In other situations, the bait and prey do interact in the assay and consequently trigger transcription of the marker gene. However, the interaction between prey and bait is physiologically irrelevant because, e.g., the interaction either does not occur in vivo in the therapeutic target cell (e.g., the host cell used in the phenotypic assay) or does not play a role in the physiological pathway relevant to the phenotype under study in the therapeutic target cell (a “non-relevant interaction”).

The yeast two-hybrid technique can be adapted for high throughput protocols. Specifically, this screening technique can be adapted for the management of large sample numbers with minimal handling, in theory permitting rapid and efficient isolation of putative binding partners. This very advantage of the two-hybrid technique, however, disadvantageously magnifies the number of putative interactions from which false positives (both artifactual interactions and non-relevant interactions) must be winnowed by time-consuming individual assays or secondary screening steps.

Researchers have attempted to mitigate the false positive problem in yeast two-hybrid assays, but to date such work has focused largely on the first source of false positives—artifactual interactions (i.e., putative binding events that appear to occur in the yeast assay system but which do not occur in the endogenous cellular environment of the target cell). Such artifacts arise from a variety of factors, including oversensitivity of the yeast assay system, presence of “sticky” proteins that evidence nonspecific interactions with random molecules, self-activating molecules, and transcriptional moieties that bind DNA even absent an interaction with a second protein-binding moiety. Approaches to mitigating these artifacts include: (1) replica plating of candidate binding partners (fused to the activation domain or “AD”) with a variety of test fusion proteins on the binding domain (“BD”) moiety, with subsequent elimination of binding partners that interact with other test fusions; (2) modifying the vectors that contain the prey and bait (e.g., Louvet O. et al., Biotechniques 23(5):816-18, 820 (1997)); (3) re-engineering the host yeast cells used in the assay (e.g., Feilotter, H E et al., Nucleic Acids Res. 22(8):1502-3 (1994)); and (4) coimmunization and colocalization with an epitope-tagged protein (Wong, C. and Naumovski, L., Anal. Biochem. 252(1):33-39 (1997)). An approach utilizing dominant negative phenotypes to confirm interrelation of known gene products in yeast cells also has been described. (He and Jacobson, Genes Dev. 9(4):437-54 (1995)).

None of the prior art methods provide an effective, generally applicable method for improving the speed and accuracy of protein interaction screening. For example, approaches that eliminate artifactual interactions (e.g., replica plating) may be quite time-consuming and laborious, do not cull out physiologically non-relevant interactions, and may even eliminate some true positives. Moreover, even the use of a perturbagen as one component of a protein interaction assay does not preclude detection of binding events that ultimately are found to be unrelated to an endogenous pathway of interest.

Accordingly, an unmet need exists for reducing or eliminating physiologically irrelevant false-positives from protein-ligand interaction assays, thus streamlining the drug discovery process. Preferably, any solutions to this problem should be compatible with high-throughput screening techniques.

### SUMMARY OF THE INVENTION

The present invention provides methods for screening for physiologically relevant intermolecular interactions. These interactions often are between an endogenous protein or other proteinaceous molecule (referred to herein as an “endogenous protein”) and one or more corresponding ligands. Such endogenous protein-ligand interactions often participate in or indirectly affect an endogenous cellular pathway of interest. Such physiologically relevant protein-ligand interactions are detected by using two independent phenotypic probes to identify and eliminate non-relevant interactions. The methods are particularly valuable for assays involving endogenous mammalian proteins, and for streamlining and focusing high-throughput screening procedures.

The inventive methods screen for physiologically relevant protein interactions by utilizing more than one independent phenotypic probe to eliminate false positives. The inventive methods do so by (i) detecting the interaction between an endogenous cellular component and a primary phenotypic probe, and (ii) determining whether the endogenous cellular component identified thereby (the “putative therapeutic target molecule”) interacts with a second, independent phenotypic probe that provides confirmation of the physiological relevance of the target. The interactions between probes and endogenous cellular components may be detected using standard protein-ligand interaction assays—e.g., the yeast two-hybrid technology. Both probes are “phenotypic” because, by interacting with an endogenous cellular component, each causes an alteration in the same (or closely related) “phenotype of interest.” The phenotype of interest, in turn, is a detectable cellular characteristic that is an indicator of the state of an endogenous genetic pathway within a cell (e.g., a biochemical/physiological pathway that provides cell-type or cell-state specific indices such as cell growth/arrest, cell metabolic state, or cellular expression of genes known to relate to the desired endogenous physiological pathway). Alteration in the phenotype of interest can be detected either directly (e.g., as in the case of growth), or indirectly (e.g., through alteration in the expression pattern of a reporter that correlates to that phenotype.) In some embodiments of the invention, the results of the first phenotypic interaction are used to force the second round of protein interaction and phenotypic assays to converge upon a smaller, more focused group of phenotypic probes.

The above-summarized methodology provides a parallel screening protocol for establishing the physiological relevance of putative therapeutic targets. First, testing with at least two “independent” probes—i.e., probes that are identified in separate assays, and which can be optionally derived from a separate library—reduces or eliminates false positives that derive from artifactual interactions. Second, testing with at least two “phenotypic” probes substantially increases the likelihood that the binding partner is physiologically relevant, because interaction with more than one probe that causes an alteration in the same (or closely related) phenotype of interest provides strong validating evidence that the protein-ligand interaction is in fact linked to the endogenous cellular pathway(s) related to the phenotype. Because both the first and subsequent probes are independently shown to be physiological effectors of the same or related phenotypic trait, any endogenous cellular component that interacts with both probes is highly likely to be a true positive—i.e., to be involved in a physiologically relevant endogenous cellular pathway in the cell. When the phenotype of interest is selected so as to relate to, e.g., a disorder or disease of interest, the inventive methodology provides strong evidence that any endogenous cellular component thus identified is a validated therapeutic target. That validated target, in turn, has many uses, including (i) screening for small molecules that bind to the target and exert a therapeutic effect, (ii) elucidating physiological pathways, (iii) identifying gene(s) that encode or relate to the target, and (iv) providing the basis for diagnosing related physiological abnormalities.

In particular aspects of the claimed invention, the phenotypic probes will be “perturbagens.” The nature and use of perturbagens have been described in more detail in co-pending, co-owned applications U.S. Ser. No. 08/699,266, filed Aug. 19, 1996 (“Selection Systems For The Identification Of Genes Based On Functional Analysis”), WO98/07886, and in U.S. Ser. No. 08/812,994, filed Mar. 4, 1997 (“Methods For Identifying Nucleic Acid Sequences Encoding Agents That Affect Cellular Phenotypes”), the disclosures of which each are specifically incorporated by reference in their entirety. Succinctly, perturbagens include proteinaceous molecules (proteins, protein fragments or domains, polypeptides or peptides) or nucleic acid moieties that act in a transdominant mode by interacting with endogenous components of a target cell (rather than on alleles of genes), and thereby interfering with normal cellular function. The perturbagens typically interact with proteins or polypeptides that reside in or on the therapeutic target cell. That therapeutic target cell is often a mammalian cell, in some embodiments, a human cell that is cancerous or virally infected.

Certain aspects of the inventive methods feature the yeast two-hybrid assay system, although the claimed inventions are generally applicable to other methodologies for detecting protein-ligand interactions. In one exemplary embodiment, at least two rounds of protein interaction assays and two independent sets of phenotypic probes are used to validate the physiological significance of any putative endogenous target molecule. In this particular embodiment, one cycles between verifying the physiological significance of a perturbagen or other such probe, and identifying endogenous proteinaceous components that bind to those physiologically relevant probes. Optionally, the “prey” or interaction target used in the first yeast two-hybrid assay may be used as the “bait” or interaction probe in a subsequent yeast two-hybrid assay step. The basic inventive method may also include an additional step of counter-selecting against interaction probes that self-activate. This additional step provides still further advantageous elimination of false positives that are assay artifacts.

With the present invention, it is possible to identify protein interactions based on a phenotype at the outset, and further test these interactions en masse to pinpoint the true, physiologically relevant interactions.

### BRIEF DESCRIPTION OF THE DRAWINGS

FIG. 1 is a pictorial flow chart summarizing the basic methodology of identifying physiologically validated target molecules with phenotypic probes.

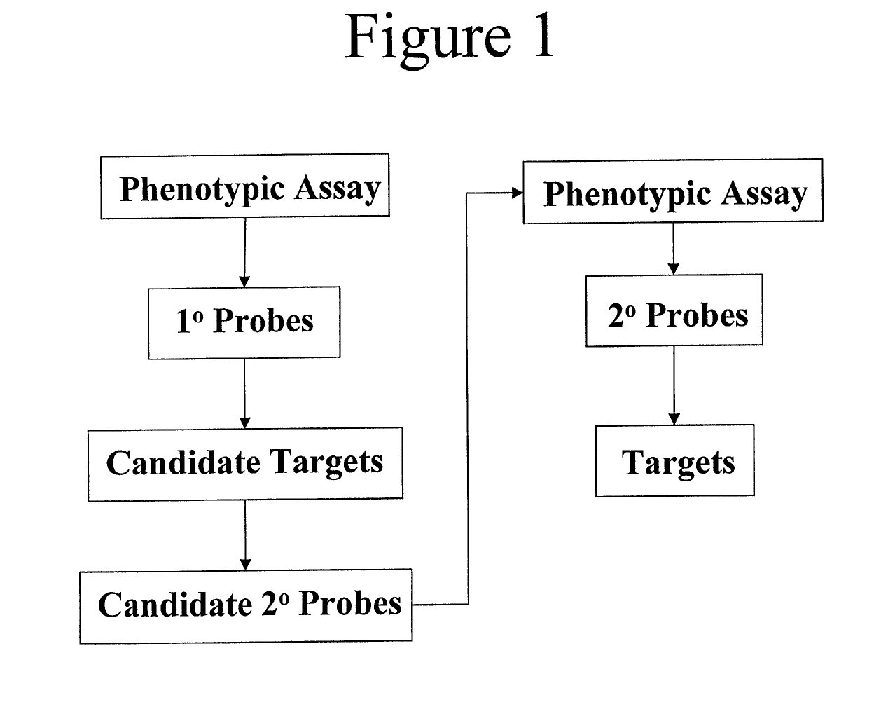

FIG. 2 is a pictorial flow chart summarizing a method of identifying physiologically validated target molecules utilizing phenotypic probes (identified with physiological assays) and yeast two-hybrid protein interaction assays.

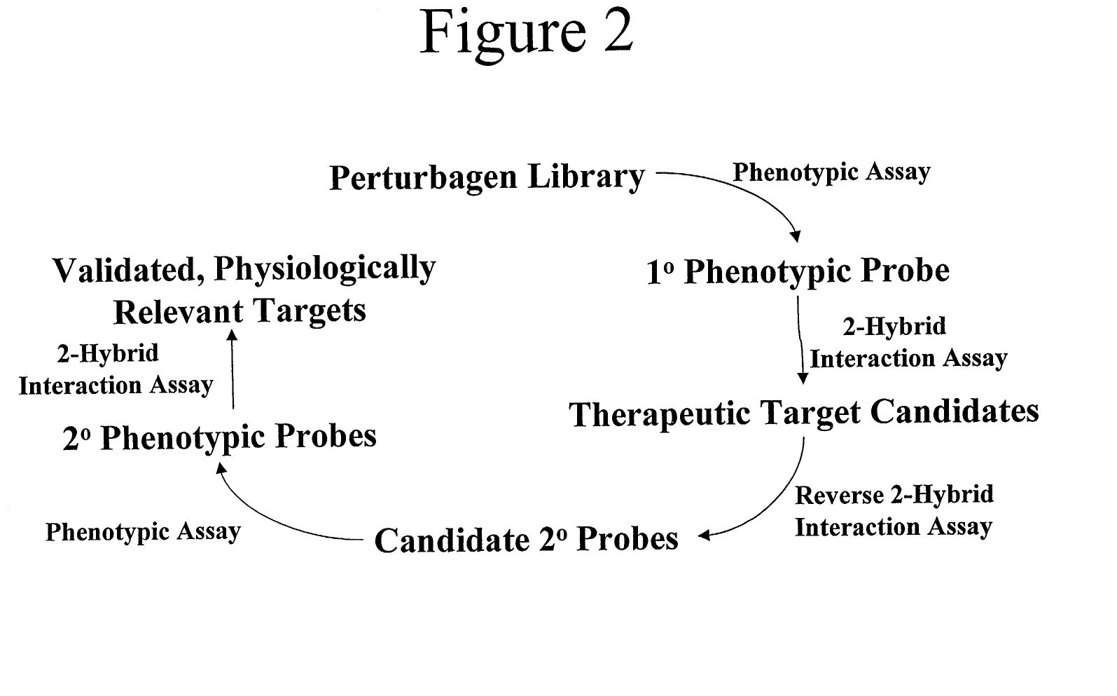

FIG. 3 is a diagram of representative yeast two-hybrid reporter constructs that are designed for use in a Gal4-based reporter system: (1) pVT85 (The shaded region represents the upstream activating sequence (UAS) and part of the 5′ untranslated region (UTR) of the yeast Gal2 gene spanning nucleotides 9-854 5′ of the Gal2 gene. The open box represents the first 9 nucleotides of the 5′ UTR, entire coding region and first 81 nucleotides of the 3′ UTR of the URA3 gene. Regions denoted with single lines represent chromosome 2 DNA flanking the reporter ending at nucleotide 473885 (5′ region) and starting at nucleotide 469705 (3′ region)); (2) pVT87 (schematics as for pVT85, except that the nucleotides 9-535 5′ of the Gal1 gene is used, and the open box represents the first 10 nucleotides of the 5′ UTR and entire coding region of the His3 gene. Regions denoted with single lines represent chromosome 15 DNA flanking the reporter ending at nucleotide 721943 (5′ region) and starting at nucleotide 722607 (3′ region)); (3) pVT88 (schematics as for pVT87, except that the nucleotides 38-242 5′ of the Gal7 gene is used, and the open box represents the first 10 nucleotides of the 5′ UTR and entire coding region of the His3 gene); and (4) pVT89 (the shaded region represents the UAS, 5′ UTR and a portion of the coding region of the Gal1gene in total spanning nucleotides −535 to +87 of the Gal1 gene. The open box represents the coding region of the LacZ gene fused to the Lys2 3′ UTR).

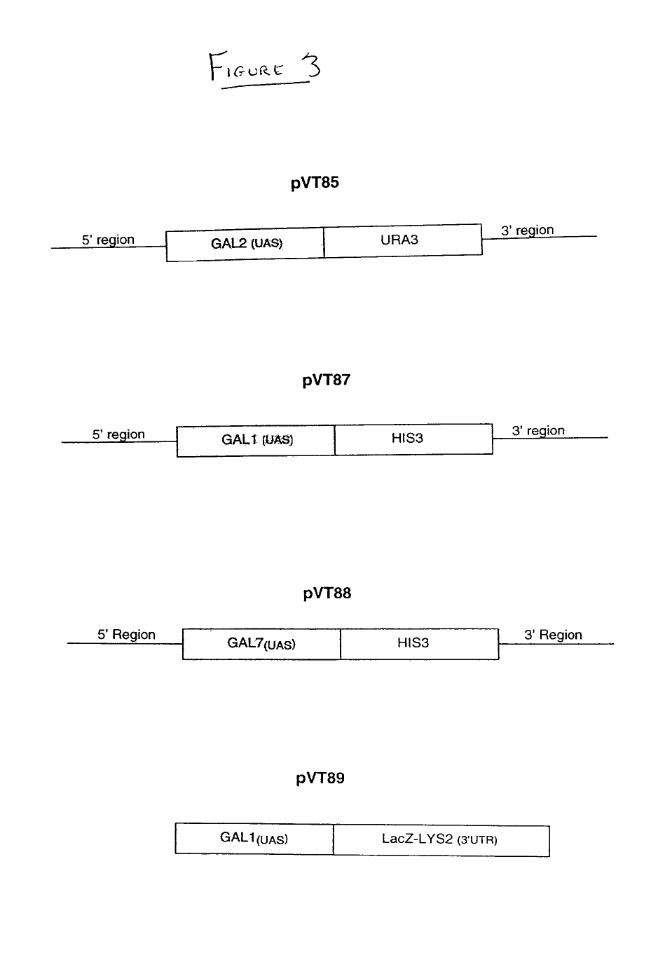

FIG. 4 is a diagram of representative yeast two-hybrid reporter constructs that are designed for use in a LexA-based reporter system: (1) pVT86 (the shaded region denotes eight LexA operators embedded within the Gal1 UAS; the open box represents the first 9 nucleotides of the 5′ UTR, entire coding region and first 81 nucleotides of the 3′ UTR of the Ura3 gene. Regions denoted with single lines represent chromosome 2 DNA flanking the reporter ending at nucleotide 473885 (5′ region) and starting at nucleotide 469705 (3′ region); and (2) pVT90 (schematics at in pVT86, but the open box represents the LacZ gene).

### DETAILED DESCRIPTION OF THE PREFERRED EMBODIMENTS

Overview of the Screening Methodology

Developing new therapeutic agents and identifying genes that are involved in disease pathways share a common prerequisite—the need to delve into the molecular workings of the therapeutic target cell (e.g., a cancer cell) and to identify endogenous cellular components that are suitable targets for further research. These cellular components may be part of an endogenous intracellular pathway that is related to a particular cellular abnormality, disorder or disease, for example melanoma, breast cancer, or viral infection. Alternatively, the endogenous cellular components may be cell-surface or membrane components, such as proteins, glycoproteins or phospholipids, that participate in cell-signaling, cell-recognition or other endogenous cellular pathways. Non-limiting examples of such target cellular components include: tyrosine kinases, G proteins, G protein-coupled receptors, cyclins, transcription factors and integrins. Modification or disruption of such endogenous intracellular or cell-surface pathways (collectively referred to herein as “physiological pathways”) may lead to a cellular abnormality, disorder or disease state. Thus, identifying any endogenous cellular components that are involved in or related to such physiological pathways may provide valuable insight into diagnosis or treatment.

The first step in identifying relevant endogenous cellular interactions is to select a host cell for use in an assay that is representative of a therapeutic target cell (e.g., HS294T/melanoma). DNA encoding a phenotypic probe (e.g., a perturbagen) is introduced into the assay cell line. The perturbagen expression product then specifically interacts with one or more endogenous cellular components to affect or perturb (i.e., increase, decrease or otherwise alter) the normal activity of one or more of the endogenous components in the host cell. Altering the behavior of the endogenous components may, in turn, alter or perturb a physiologically relevant pathway associated with those components. That perturbation can be detected by a correlative change in a phenotypic characteristic (also referred to as the “phenotype of interest” or “phenotypic state”) of the target cell. A “phenotypic” characteristic refers to a measurable or monitorable indicia of the physiological state or appearance of the cell. The selected phenotypic characteristic may be used directly as a precise indicator (e.g., as a screening, or preferably, a selection criterion) of the physiological state of the targeted pathway. Alternatively, the phenotypic state of the target cell may be monitored through detecting changes in the level of expression of a separate reporter gene that is not necessarily part of the physiological pathway of interest but which, nonetheless, correlates to the phenotype.

Next, endogenous cellular components that interact with the phenotypic probe are identified using standard biochemical or quasi-genetic protein interaction assay techniques (e.g., two-hybrid systems in yeast, bacteria or mammalian cells). This interaction assay completes the first round of phenotypic target screening (i.e. a first physiological assay in the target cell to identify a phenotypic probe, followed by a first protein-ligand interaction assay to identify endogenous cellular components that interact with that first phenotypic probe), and yields a first pool of interacting cellular components, termed herein “putative therapeutic target molecules” (also referred to herein as putative targets, therapeutic target candidates or the candidate target library). These putative targets may be entirely true positives (i.e. endogenous cellular components that relate to a physiological pathway of interest). Alternatively, and more likely, the set of components may contain a high percentage of false positives identified on the basis of an artifactual interaction in the protein interaction assay. Or, the set of components may contain false positives in that they are interactions that do occur in the target cell, but that do not have relevance to the endogenous physiological pathway of interest.

To segregate true positives from false positives (i.e., to identify physiologically relevant endogenous cellular interactions), another cycle of independent phenotypic evaluation is utilized to validate the physiological relevance of these putative therapeutic target molecules. Generally, in one preferred embodiment, this technique involves using standard protein-ligand interaction assays to expose the above-described putative therapeutic target molecules (i.e., the pool of endogenous molecules that interacted with the first phenotypic probe) to a second, independent pool of putative confirmatory probes (also referred to herein as candidate secondary probes)—for example, a new putative perturbagen library. (Note that in this embodiment, this second set of probes has not yet been established as “phenotypic” probes, in that the ability to perturb the host cell in an appropriate physiological assay has not yet been evaluated.) From this second protein-ligand interaction assay, those sequences encoding putative confirmatory probes (candidate secondary probes) that bind to the putative target molecules are isolated either by PCR or by plasmid isolation techniques, re-cloned into expression vectors suitable to drive expression in the host cells used in the phenotypic assay, introduced into those host cells, and subjected to another round of phenotypic assaying. This second phenotypic assay may be identical to the assay used in the first round of phenotypic screening, or it may be chosen to monitor and select a closely related phenotype of interest. This second round of phenotypic screening culls a second independent set of physiologically significant, confirmatory phenotypic probes from the sublibrary of putative confirmatory probes (candidate secondary probes) that bound to the candidate target molecule(s).

Finally, in this preferred embodiment, if the first cycle of phenotypic evaluation generated a pool of putative targets, then individual probe/target pairings may be identified by performing a third protein interaction assay. To do so, the confirmatory phenotypic probes are exposed to the library of putative therapeutic target molecules and, again using standard protein-ligand interaction techniques, members of the library of putative therapeutic target molecules that bind to particular confirmatory phenotypic probes are identified and isolated. Because binding with the putative therapeutic target molecules is used as a criterion for narrowing the pool of putative confirmatory probes that are subjected to the second phenotypic assay, the second round of screening forces a convergence upon a more focused group of secondary phenotypic probes. Alternatively, if only one individual putative target is identified by the first round of phenotypic screening, the final protein-interaction step is not required to correlate that individual phenotypic probe to the corresponding endogenous cellular binding partners.

In another embodiment, the above-described steps of (i) phenotypic screening and (ii) screening for protein-ligand interaction are reversed. Specifically, the second, independent pool of putative confirmatory probes (typically, a perturbagen library) is first phenotypically assayed to select a sublibrary of confirmatory phenotypic probes. Only then are the secondary probes exposed to the library of putative therapeutic target molecules. One may advantageously use either embodiment, based upon the relative speed and/or ease of the selected phenotypic assay protocol vs. the selected protein interaction assay protocol, and/or based on the number and binding characteristics of the perturbagens identified in the first and second rounds of phenotypic target screening.

Phenotypic Assays

Discriminating between interactions that are relevant to the endogenous physiological pathway of interest and those that are irrelevant advantageously uses probes that have phenotypic relevance—i.e., the ability to directly or indirectly correlate a phenotypic change to a particular endogenous cellular interaction by perturbing the normal physiologic function of a host cell that has been selected to represent the ultimate therapeutic target cell.

When the phenotypic change is monitored directly, the action of the phenotypic probe or perturbagen at the molecular level within the target cell results in a readily measurable or identifiable change. Such changes can include, but are not limited to; (i) cell growth in the presence of various cytotoxic or cytostatic stimuli such as, e.g., yeast mating pheromone, retinoic acid, chemotherapeutic agents like Cis-platin and growth factor deprivation such as insulin withdrawal (ii) cell death or cell cycle arrest in the presence of specific stimuli or agents such as; e.g., tumor suppressors such as p16; (iii) behavioral changes such as gain or loss of adhesion; (iv) changes in gross cellular morphology, for example alterations that are visible microscopically, or (v) directly observable changes in protein expression, e.g. cell-surface proteins.

When the phenotypic change is monitored indirectly, the phenotypic state is monitored via an appropriate surrogate—e.g., a reporter gene that correlates to but may be independent of the phenotype. Examples include, without limitation, (i) induction, in the presence of defined stimuli, of specific reporter genes (e.g., fluorescent materials such as GFP) fused to cis regulatory sequences whose expression is linked to a phenotype of interest such as apoptosis; and (ii) reduction in reporter gene expression in the presence of defined stimuli, again using a reporter whose expression is linked to the phenotype of interest. Such indirect monitoring is described in detail in U.S. Ser. No. 08/812,994, supra, incorporated herein by reference. Representative assays that monitor exemplary phenotypic characteristics are listed in Table 1.

TABLE 1
Representative Phenotypic Assays

| | Cell line used in | Therapeutic target |
|---|---|---|
| Phenotype | Assay | cell type |
| Yeast mating pheromone | Yeast | Screening model for |
| response | | growth in human cells; |
| | | model antifungal |
| | | screening system |
| FUS1-up/down regulation | Yeast | Screening model for |
| | | growth in human cells; |
| | | model antifungal |
| | | screening system. |
| P16 tumor suppressor | HS294T (human | Metastatic melanoma; |
| resistance/release from | melanoma cell line), | also a generally |
| apoptosis | and other carcinoma | applicable screening |
| | cell lines | model for other |
| | | carcinomas. |
| P16 tumor suppressor | WM35 (human | Late melanoma; also a |
| resistance/release from | melanoma cell line), | generally applicable |
| apoptosis | and other carcinoma | screening model for |
| | cell lines | other carcinomas. |
| Serum/insulin independent | WM1552C (human | Early melanoma; also |
| growth | melanoma cell line), | a model for other |
| | and other carcinoma | early-stage |
| | cell lines | carcinogenic cell lines |
| | | that are growth-factor |
| | | independent. |
| Cis-Platin resistance/ | WM35 and other | Early melanoma; also |
| selection based on cellular | carcinoma cell lines | a generally applicable |
| resistance to a model | | model for |
| chemotherapeutic agent. | | chemotherapy-resistant |
| | | carcinomas. |
| Foci development/loss of | NIH3T3 (mouse | Model for any human |
| contact Inhibition | fibroblast cell line) | cancer cell for which |
| | | loss of contact |
| | | adhesion is a |
| | | representative feature. |
| Adenovirus resistance | 293 (embryonic | Model for adenovirus |
| (growth in presence of | kidney cell line) | infections associated |
| adenovirus) | | with, e.g., the common |
| | | cold. Also a model |
| | | system for other |
| | | viruses that infect a |
| | | variety of human cells. |
| Serum-independent growth | WM1552C and | Early melanoma; also |
| (i.e., cells capable of | other Carcinoma | a model for other |
| growing without normally- | cell lines | early-stage |
| required growth factors) | | carcinogenic cell lines |
| | | that are growth-factor |
| | | independent. |
| Retinoic acid resistance | WM35 and other | Late melanoma; also a |
| (causes cell death). | Carcinoma cell lines | general model for |
| | | treatment-resistant |
| | | carcinomas. |
| Retinoic acid resistance | HS294T and other | Metastatic melanoma; |
| (causes cell death). | Carcinoma cell lines | also a general model |
| | | for treatment-resistant |
| | | carcinomas. |
| P16 up-regulation | All the above, and | Generally applicable |
| (induction of P16 tumor | other carcinoma cell | model for carcinomas. |
| suppressor expression) | lines | |
| Retinoic acid response | WM35 and other | Early melanoma; also |
| element up-regulation | Carcinoma cell lines | a general model for |
| | | treatment-resistant |
| | | carcinomas. |
| DyePKH26: a model dye | All the above, and | Generally applicable |
| that normally partitions into | other carcinoma cell | model for carcinomas. |
| the membrane during cell | lines | |
| division; detects changes in | | |
| cell division. | | |
| Caspase dyes (substrates for | All the above, and | Generally applicable |
| proteases activated during | other carcinoma cell | model for carcinomas. |
| apoptosis); detects | lines | |
| apoptosis. | | |
| Binding of Apo2.7 (an | All the above, and | Generally applicable |
| antibody that recognizes an | other carcinoma cell | model for carcinomas. |
| epitope present on dying | lines | |
| cells): detects cell death. | | |
| “Floaters” (cells that detach | All the above, and | Generally applicable |
| from the plate or other | other carcinoma cell | model for carcinomas. |
| substrate): detects cell | lines | |
| death. | | |

In some instances, the cell line used in the phenotypic assay (also termed herein the “phenotypic assay host cell” or simply “host cell”) may actually be the identical to the therapeutic target cell (i.e., the phenotypic assay may be performed directly on the cancer cells of interest). In other instances, the phenotypic assay host cell may be of the same origin as the therapeutic target cell, but is modified in some way for laboratory use. In still other instances, the phenotypic assay host cell may be selected so as to be representative of some aspect of the therapeutic target cell, but is unrelated to that cell (e.g., using yeast cells to model human cells, or using embryonic kidney cell line 293 as a model for viral infection of mammalian cells in general). In any event, a wide variety of suitable phenotypic assays and host cells are known to those of skill in the art, and Table 1 is only a representative sampling of such cells and assays.

Phenotypic Probes

An exogenous molecule that effects a change in the phenotype of interest in a selected host cell is termed a phenotypic probe. As explained in U.S. Ser. Nos. 08/699,266 and 08/812,994, supra, perturbagens are a class of molecules with great utility as phenotypic probes. As such, perturbagens interact with the endogenous physiological pathways of a cell and cause correlative phenotypic changes that are useful surrogates for tracking disruption of the endogenous pathways within the target cell. As described in more detail in the above-referenced, related applications and elsewhere herein, these phenotypic changes are detected using appropriate assays.

Perturbagens may be proteinaceous molecules (proteins, protein fragments or domains, polypeptides or peptides), nucleic acid moieties that interact with endogenous components of a target cell, or other organic or inorganic compounds. Proteinaceous perturbagens may be presented to the system of choice as products of expression libraries comprised of, e.g., synthetic DNA, cDNA or fragmented, sheared or digested genomic DNA (“perturbagen libraries”). Perturbagens may be expressed in cells without any additional sequences joined to them, or alternatively may be fused to other molecules. For example, a polypeptide may be fused to the perturbagen to increase stability of the perturbagen in the assay system and/or to provide an easily detectable feature, such as fluorescence. Examples of such fusion moieties include GFP, LacZ or Gal4. Details are provided in co-pending, co-owned U.S. Ser. No. 08/965,477, “Methods And Compositions For Peptide Libraries Displayed On Light-Emitting Scaffolds,” the disclosure of which is incorporated herein in its entirety.

Once expressed within the target cell, perturbagens may induce a phenotypic state in the host cells that tracks or mimics a genetic mutant or epigenetic state. Any alteration in the target cell is detected through monitoring correlative changes in a reporter gene, or in another appropriate characteristic such as cellular growth or morphology, or expression of a marker. If a reporter gene is used, it is chosen to correlate with and thus reflect the relevant phenotypic state as closely as possible. The reporter is expressed in the host cells at a level sufficient to permit its rapid and quantitative determination.

Disruption of Previously Unidentified Endogenous Pathways and Components

The methods of the invention preferably may advantageously be applied to identify components of previously uncharacterized biochemical or physiological pathways in the target cell, for example, genetic discoveries of pattern formation genes in Drosophila, C. elegans, and mammals. In other embodiments, the methods of the invention may advantageously be applied to isolate a previously unidentified endogenous component of an endogenous pathway that previously had been at least partially characterized. Examples include using revertants from p16-mediated cell cycle arrest to identify downstream components of the p16 pathway involved in growth control.

Reporter Genes

Numerous reporter genes have been appropriated for use in expression monitoring, and are thus suitable for indirect monitoring of the phenotypic state of the cell. A reporter comprises any gene product for which screens or selections can be applied. Reporter genes used in the include the LacZ gene from E. coli (Shapiro S. K., Chou J. et al, Gene November; 25: 71-82 (1983)), the CAT gene from bacteria (Thiel G., Petersohn D., and Schoch S., Gene February 12; 168: 173-176 (1996)), the luciferase gene from firefly (Gould S. J. and Subramani S., 1988), the GFP gene from jellyfish (Chalfie, M. and Prasher D. C., U.S. Pat. No. 5,491,804), and modified or mutated forms of GFP (Abedi et al (1998)). This set has been primarily used to monitor expression of genes in the cytoplasm. A different family of genes has been used to monitor expression at the cell surface, e.g. the gene for lymphocyte antigen CD20. Normally a labeled antibody is used that binds to the cell surface marker (e.g., CD20) to quantify the level of reporter (Koh, J., Enders, G. H. et al., 1995).

Native GFP is a member of a family of naturally occurring fluorescent proteins, whose fluorescence is primarily in the green region of the spectrum. Native GFP has been developed extensively for use as a reporter and several modified or mutant forms of the protein have been characterized that have altered spectral properties (e.g., Cormack, B. P., Valdivia R. H. and Falkow, S., Gene 173: 33-38 (1996)). (Both native GFP and such related molecules are collectively referred to herein as “GFP”) High levels of GFP expression have been obtained in cells ranging from yeast to human cells. It is a robust, all-purpose reporter, whose expression in the cytoplasm can be measured quantitatively using a flow sorter instrument such as FACS.

Of these reporters, autofluorescent proteins (e.g., GFP) and the cell surface reporters are potentially of greatest use in monitoring living cells, because they act as “vital dyes.” Their expression can be evaluated in living cells, and the cells can be recovered intact for subsequent analysis. Vital dyes, however, are not specifically required by the methods of the present invention. It is also very useful to employ reporters whose expression can be quantified rapidly and with high sensitivity. Thus, fluorescent reporters (or reporters that can be labeled directly or indirectly with a fluorophore) are especially preferred. This trait permits high throughput screening on a flow sorter machine such as a fluorescence activated cell sorter (FACS).

The selected reporter gene may also advantageously act as a scaffold for a desired sequence (e.g., DNA encoding a perturbagen). GFP, for example, can be used as such a scaffold, and the structure of the expressed polypeptide serves as a stabilizing polypeptide for the perturbagen insert. The perturbagen sequence can either be inserted at or near the N- or C-terminus of the GFP scaffold, or alternatively can be inserted into a suitable internal site. The use of GFP as a stabilizing polypeptide scaffold is described in U.S. Ser. No. 08/965,477, supra, and is incorporated by reference herein.

High-Throughput Protocols

Preferably, individual cell phenotype states are determined via a selection device or method that permits rapid, quantitative measurement of the expression levels of the reporter, selection molecule or other selection criterion on a cell-by-cell basis. As used herein, the phrase “high throughput” refers to cell sorts of at least 1×10³ cells per hour, and more preferably 1×10⁷ cells per hour. High throughput screens, selections or assays generally involve techniques that permit numerous cells or reactions to be analyzed either in parallel, or in a rapid serial fashion. For example, the flow sorter is a high throughput serial device since it can examine roughly 1×10⁸ cells per hour.

In one preferred high-throughput embodiment, cells with a desired phenotypic profile are isolated, for example, by flow cytometry or other appropriate separation technique. Cell separation may be performed on the basis of any suitable selection criteria, such as fluorescence or magnetic characteristics. The resident probes that correlate to that phenotype are recovered, for example by PCR amplification of the resident DNA sequences that encode them. These recovered probes, in turn, are used to isolate their endogenous binding partners. Generally, this can be accomplished using standard protein interaction techniques—for example, by biochemical binding assays or yeast two-hybrid assays. DNA encoding a pool of putative therapeutic target molecules that are perturbagen binding partners is then isolated, for example by standard techniques of DNA recovery followed by PCR amplification using flanking sequences as primer-binding sites, or by plasmid isolation techniques familiar to those skilled in the art.

Embodiments Using Yeast Two-Hybrid Technology

The phenotypic evaluation steps of the present invention require some method for detecting interactions between proteinaceous perturbagen probes and endogenous target molecules. The yeast two-hybrid technique is one such method. When the yeast two-hybrid technology is applied in the context of the present invention, it may be used to detect interaction between putative or actual phenotypic probes and putative or actual endogenous therapeutic targets. The general strategy and experimental details of this method are familiar to those of ordinary skill in the art.

In this embodiment, a population of putative endogenous therapeutic target molecules is identified with a first set of phenotypic probes (e.g., perturbagens) by first introducing the initial library of putative perturbagen-encoding sequences into the cell line used in the phenotypic assay (e.g., human cell lines HS294T, WM35, or WM1552C—representative of human melanoma therapeutic target cells). The cells containing the perturbagen DNA are then subjected to a phenotypic selection or assay, such as a protocol for selecting variant cells that grow in the presence of a stimulus that kills the vast majority. Cells having the desired phenotype are identified and segregated via standard techniques such as FACS or growth-based characteristics. From these phenotypically culled cells, a primary set of phenotypic probes that altered the physiological state of the cells are recovered. The phenotypic probe is presumed to have exerted its phenotypic effect through an interaction with a relevant endogenous component of the target cell.

Next, a first yeast two-hybrid protein interaction assay is performed to identify a pool of endogenous cellular components from the phenotypically culled cells that interact with the first set of phenotypic probes. These cellular components are thus identified as putative therapeutic target molecules.

As one non-limiting example, each of the physiologically relevant primary perturbagens, described above, is cloned as a fusion with the DNA binding domain of a transcription factor, e.g., Gal4, as the bait or interaction probe for a two-hybrid search. These phenotypic bait constructs are introduced into an appropriate yeast strain, for example any strain suitable for co-transformation, or having the ability to mate to yeast cells of the opposite mating type and capable of serving as a vehicle for propagation of and selection for the transcription factor (e.g., Gal4) fusion bait-encoding plasmid. Next, this strain is mated to a second strain of yeast that harbors the prey library that has been cloned in frame with an appropriate AD. As one non-limiting example, that library is constructed so as to contain all possible protein domains present in the target cell or organism of interest. This can be accomplished (in whole or in large part) by the use of fragmented gDNA or random-primed cDNA cloned into the appropriate yeast two-hybrid expression vector.

The yeast cells containing the prey constructs are then mated to yeast cells that harbor the perturbagen-containing bait constructs. The resultant mated cells are plated on an appropriate medium (e.g., a medium designed to detect the particular marker activity that is associated with the AD/BD complex). Yeast cells expressing the marker gene are recovered. An unspecifiable portion of these cells will evidence marker gene expression due to interaction between the bait (perturbagen) and the prey (endogenous cellular target candidates). These specific prey sequences comprise a sub-library of candidate perturbagen binding partners or targets. The candidate target sub-library can be recovered as pure DNA (absent the yeast) by PCR amplification using flanking sequences as primer-binding sites, or by plasmid isolation techniques familiar to those skilled in the art. Alternatively, the bait and prey constructs can be introduced into the same yeast cell and the resultant co-transformed yeast cells are plated on medium with recovery of cells expressing the marker gene.

These candidate target sequences may be amplified by reintroduction of the plasmids into E. coli, or by PCR. They may then be re-cloned as bait sequences (e.g., using the original GAL4 BD domain as a fusion partner) in preparation for a second round of yeast two-hybrid screening. Alternatively, the initial yeast two-hybrid screen can be performed in a “backwards” context in which the bait is linked to the AD domain and the prey (candidate target library) is linked to the BD domain. This obviates the need for a subsequent switch involving a re-cloning step to shuttle the prey into the bait vector. Instead, the initial candidate target sequences are recovered in an AD vector, and these can be used directly to screen a second library in a BD vector.

Optionally, self-activating sequences may be depleted from the second BD-fusion library prior to screening that library with the selected AD-fusion library. This can be accomplished using a negative selection, e.g., a selection against a URA+ phenotype. The purpose of the negative selection is to remove from the second library sequences that self-activate; i.e., that can confer a URA+ reporter phenotype in the absence of a second interacting protein that brings in the AD fusion. The sub-library, now depleted of self-activating sequences, can then be used as the prey in a second screen using the candidate targets as bait in order to identify secondary binders that are candidate perturbagens.

Regardless of the precise composition of the prey and bait constructs, a second protein interaction assay proceeds by mating appropriate yeast host cells in order to expose the bait and prey constructs. If, for example, the set of targets has been reconstituted as fusions to the bait moiety, they are mated en masse to a second prey library which may, e.g., contain perturbagen peptide sequences fused to the Gal4 activation domain. Once again, the transformed yeast are plated onto selective medium appropriate for the marker gene responsive to the BD/AD interaction construct, and pairs of target/prey interactors are recovered.

From this pool of second binding partners, a set of putative confirmatory probes (also referred to herein as candidate secondary probes) is recovered. These probes are PCR-amplified and cloned into a mammalian expression vector, for example a CMV-derived vector. The probes are then introduced into suitable host cells, for example those used in the original physiological assay, and subjected to a physiologic selection or screen in order to select a second pool of phenotypic probes. Those secondary phenotypic probe sequences that confer the same or similar physiological effect on the host cells as the original perturbagens (i.e., generate the phenotype of interest) are recovered. Finally, these secondary phenotypic probes are used to validate the physiological significance of members of the candidate target library. Candidate targets that bind to both the primary and secondary phenotypic probes are true targets. Because these endogenous targets are now shown to interact with two separate, independent sets of phenotypic probes, the target is an overwhelming choice for an in vivo therapeutic target.

The logical basis for matching a particular perturbagen to a particular target protein involves the identification of two independent effectors (e.g., perturbagens) that confer identical or similar physiological changes on host cells, and recognize the same target protein in protein-ligand interaction assays such as the yeast two-hybrid system. Using the series of steps described herein, it is possible to find two perturbagens that bind the same target protein, because the protein-ligand interaction steps force the perturbagens to converge on the same set of candidate targets (i.e., the second confirmatory effector perturbagen is isolated based on its ability to bind to a binding partner of the first perturbagen). In addition, the second confirmatory perturbagen (as well as the first) are identified by their physiological effect on cells. Thus, it becomes exceedingly unlikely that the common target of the two perturbagens is not the physiologically relevant target.

Yeast Two-Hybrid Reporter Constructs

The yeast two-hybrid reporter gene is typically fused to the upstream promoter region that is recognized by the BD, and is selected to provide a marker that facilitates screening. Examples include the lacZ gene fused to the Gal1 promoter region and the His3 yeast gene fused to Gal1 promoter sequences. A variety of yeast two-hybrid reporter constructs are suitable for use in the validation methods of the present invention. Desirable criteria for these reporter constructs are that they provide a rigorous selection (i.e., yeast cells die in the absence of a protein-ligand interaction between the bait and prey sequences), or a convenient screen (e.g., the cells turn color when they harbor bait and prey sequences that interact). Examples include (1) the Ura3 gene, which confers growth in the absence of uracil and death in the presence of 5-fluoroorotic acid (5-FOA); (2) the His3 gene, which permits growth in the absence of histidine; (3) the LacZ gene, which is monitored by a colorimetric assay in the presence/absence of beta-galactosidase substrates; (4) the Leu2 gene, which confers growth in the absence of leucine; and (5) the Lys2 gene confers growth in the absence of lysine or, in the alternative, death in the presence of α-aminoadipic acid. These reporter genes may be placed under the transcriptional control of any one of a number of suitable cis-regulatory elements, including for example the Gal2 promoter, the Gal1 promoter, the Gal7 promoter, or the LexA operator sequences.

Yeast Two-Hybrid Host Strains

A variety of yeast host strains known in the art are suitable for use in the validation methods of the present invention. Desirable criteria for these host strains are that they can be mated to cells of opposite mating type (i.e., they are haploid), and they contain chromosomally integrated reporter constructs that can be used for selections or screens (e.g., His3 and LacZ). Generally, either Gal4 strains or LexA strains may be used with the appropriate reporter constructs. Examples include strains yVT96, yVT97, yVT98 and yVT99, described herein. Additionally, those of ordinary skill will appreciate that the host strains used in the present invention may be modified in other ways known to the art in order to optimize assay performance. For example, it may be desirable to modify the strains so that they contain alternative or additional reporter genes that respond to two-hybrid interactions.

Embodiments Using Biochemical Binding Assays to Detect Interactions.

As an alternative to using quasi-genetic methods such as the yeast two-hybrid methodology for detecting protein-ligand interactions, biochemical methods may be used to detect targets and to identify the second candidate perturbagens. For example, affinity purification techniques are well known to those of skill in the art. Proteinaceous probes such as perturbagens may be used as one component of an affinity purification, specifically to select perturbagen binding partners from a cellular extract. The perturbagens and associated endogenous cellular binding partners are isolated and collected for analysis by standard analytical methods. As one non-limiting example, mass spectrometric methods may be used to separate and characterize the endogenous perturbagen-binding proteins. By reference to sequence databases, the identity of the binding partner can be identified. This in turn facilitates isolation of cDNA encoding the binding partner for expression of suitable amounts of purified protein for use in a standard phage display procedure (e.g., expressed on phage). The purified candidate targets are exposed to a second set of candidate confirmatory probes. Probes from the phage display library that bind to the purified protein are recovered and subjected to an appropriate physiological assay, as described above. Finally, phenotypically relevant confirmatory probes are recovered as above. Candidate endogenous cellular targets that bind to these probes are identified and isolated, as above.

Advantages of the Validation Methodology

The parallel phenotypic validation strategy of the present invention provides a powerful tool for screening potential therapeutic agents for their ability to effect a desired change in a physiologically relevant pathway.

One important feature of the invention described herein is that a particular putative therapeutic target molecule, known from protein-ligand interaction assays to interact with a perturbagen probe, can be linked with a high degree of certainty to a defined physiological pathway. Thus, it is possible to relate protein-ligand interactions to physiological pathways in cells, a link that is very difficult and time-consuming to establish normally. Without the approach described herein, each candidate target must be tested independently and painstakingly for a physiological role. This requires, for example, the production of antibodies or antisense constructs, their introduction into cells, and the monitoring of specific phenotypes.

The protocols of this invention are very advantageous because they permit high-throughput screening for endogenous targets of specific peptide or protein effectors that alter cellular physiology in defined ways. The specific advantages are twofold: first, the screening can be carried out en masse, obviating the need to painstakingly examine each candidate target individually. Second, false positives (e.g., spurious protein-ligand interactions identified via protein interaction assays) can be readily reduced or even eliminated. These advantages have important consequences. They sidestep a major obstacle in the upstream portion of the drug development process; namely, the difficulty of identifying validated, true targets of effector molecules (e.g., perturbagens). This is accomplished by tying specific perturbagen binding partners to physiological roles in the cell; that is, linking specific cellular proteins to definite biochemical/physiological pathways in cells.

It should be borne in mind that one of the major shortcomings associated with genomics and proteomics methods at present is the extreme difficulty associated with matching particular genes or proteins with physiological roles in cells. The methods described here provide a significant contribution to the solution to this problem. Using this technology, protein-ligand interactions can be assigned to specific physiologically relevant (and hence, medically relevant) pathways, and not merely catalogued.

The methods described herein thus provide a substantial advantage over the methodologies previously known to the art. Because any putative target candidate is linked to an endogenous cellular/physiological pathway of interest, which in turn is associated with a particular cellular abnormality, disorder or disease, its therapeutic utility is validated. This validation step provides additional efficiencies by reducing the size of the ultimate pool of targets that are to be subjected to additional research.

### EXAMPLE 1

### Creation and Characterization of the Phenotypic Probe Libraries and Candidate Target Libraries

(1) Construction of Perturbagen Libraries for Phenotypic Assays.

Phenotypic assays may often utilize libraries of putative perturbagens which are constructed so as to provide the desired variety of genetic material for screening, in a vector that is suitable for the target cell used in the phenotypic assay. For example, when the therapeutic target cell of interest is a mammalian cell, or even more particularly a human cancer cell, the library must be constructed in a manner that allows for (1) introduction of the perturbagen library into the mammalian cell and (2) subsequent expression of the library in the mammalian target cell.

As one non-limiting example, a cDNA library that encodes potential perturbagens may be prepared according to the following procedure, using methods that are well known in the art. Double-stranded DNA is prepared from random primed mRNA isolated from a particular cell type or tissue, for example human placental tissue. Alternatively, randomly sheared genomic DNA fragments may be utilized. In either case, the fragments are treated with enzymes to repair the ends and are ligated into a suitable retroviral or episomal expression vector suitable for expression in, e.g., mammalian cells. One exemplary vector is pVT334 (described in WO 98/##,### [filed Nov. 5, 1998 as the PCT counterpart of priority document Ser. No. 08/965,477], the disclosure of which incorporated herein by reference), a retroviral vector that permits the expression of library clones as EGFP fusions from the CMV promoter. Such vectors can be packaged by standard procedures into infectious particles to facilitate introduction into human cells. The perturbagen-containing vectors are then introduced into E. coli and clones are selected. A number of individual clones sufficient to achieve reasonable coverage of the mRNA population (e.g., one million clones) is collected, and grown in mass culture for isolation of the resident vectors and their inserts. This process allows large quantities of the library DNA to be obtained in preparation for subsequent phenotypic screening and protein interaction assays, as described infra.

As a second example, a synthetic DNA library of potential perturbagens of varying sizes can be prepared. For example, libraries of synthetic 15 amino acid (aa) peptides were created using the general method described in Abedi et al., Nucleic Acids Res. 26(2):623-630 (1998), and as described in co-pending U.S. patent application Ser. No. 08/965,477, supra, incorporated herein by reference. Briefly, DNA encoding randomly generated 15 amino acid peptides was synthesized and inserted into the XhoI and BamHI sites of a selected EGFP construct. These steps thus can create random peptide display libraries. Alternatively, targeted or engineered synthetic DNA libraries encoding “smart” perturbagens can be constructed. For example, a variety of DNAs encoding engineered variants of a known perturbagen may be readily constructed.

(2) Construction of Target Cell-Specific Genetic Libraries.

The protein interaction portion of the target validation methodology described herein requires presentation of a phenotypic probe to a library of proteins. In some embodiments, the proteins of interest may be particular to a selected target cell. In such cases, it is desirable to create and test a collection of endogenous cellular proteins derived from a cell line that is representative of the therapeutic target cell—e.g., HS294T, WM35 or WM1552C (melanoma). These endogenous proteins may be readily obtained by expressing a genetic library that is derived from the selected cell line. As one non-limiting example, the mRNA of the therapeutic target cell line is used to construct the therapeutic target library. Alternatively, cDNA libraries derived from fetal brain, liver or kidney may be prepared. The details of library construction, manipulation, and maintenance are as described above for the construction of a perturbagen cDNA library.

### EXAMPLE 2

### Creation and Characterization of Exemplary Yeast Two-Hybrid Assay Components

Preparation of various yeast two-hybrid assay components—e.g., bait constructs, prey constructs, and host cells—are familiar to the art. The following are exemplary, non-limiting examples of such components.

(1) Suitable Yeast Vectors

Once the phenotypic probe (perturbagen) and target libraries are selected, each is incorporated into an expression vector that is appropriate for use in yeast. The target and perturbagen libraries are deployed as bait/prey libraries in appropriate bait and prey fusion constructs.

One exemplary binding domain vector is pVT560, which has a GFP scaffold protein with internal BamHI and XhoI sites for subsequent cloning of either the perturbagen or target sequences. The pVT560-based libraries are transformed into appropriate yeast strains, for example yVT99 or yVT100. Optionally, the bait construct may be subjected to an additional step to eliminate self-activating sequences; e.g., yeast expressing peptides which self-activate transcription are removed via negative selection in the presence of 5-FOA creating a sub-library of yeast expressing non-activating sequences.

Suitable activation domain vectors include pVT563 and pVT561, which have a GFP scaffold protein with internal BamHI and XhoI sites for subsequent cloning of either the perturbagen or target sequences. The pVT563 and pVT561-based libraries are transformed into appropriate yeast strains, for example yVT97 and yVT98, respectively.

Identification of peptides capable of binding to perturbagen-target candidates is accomplished by mass-mating the peptide library expressing yeast with target-protein expressing yeast and selecting for growth on plates lacking histidine, leucine or uracil (depending on the selected reporter).

(2) Construction of Perturbagen Libraries for Yeast Two-Hybrid Assays

As described in the previous Example, perturbagen libraries may be derived from a number of sources, including without limitation synthetic DNA inserts, gDNA or cDNA, and may be inserted into a scaffold protein, for example native or modified GFP. In order to screen the perturbagen library in a yeast two-hybrid assay, it must be incorporated into a suitable vector.

The vectors pVT560, pVT561 and pVT563 were constructed as follows. Plasmid vector pVT560 was constructed by filling the BamHI and XhoI sites in pLexA (Clontech 98/99 p. 89) in separate steps using Klenow fragment. EcoRI was used to clone a GFP gene containing internal XhoI and BamHI restriction sites (as in pVT27, described in U.S. Ser. No. 08/965,477, supra, incorporated herein by reference) into the modified pLexA. The reading frame of GFP was such that it was in frame with the DNA binding domain in pLexA. Finally, a 1.2Kb BamHI-XhoI stuffer fragment (containing 1194 coding bases of the yeast STE4 gene) was cloned into the GFP gene to yield pVT560. Plasmid pVT561 was constructed beginning with pB42AD (Clontech 97/98, p. 50) as follows. The BamHI and XhoI sites in pB42AD were filled in separate steps using Klenow fragment. EcoRI was used to clone a GFP gene containing internal XhoI and BamHI restriction sites (as in pVT27, supra) into the modified pB42AD. The reading frame of GFP was such that it was in frame with the DNA activation domain in pB42AD. Finally, a 1.2Kb BamHI-XhoI stuffer fragment (containing 1194 coding bases of the yeast STE4 gene) was cloned into the GFP gene to yield pVT561. Plasmid pVT563 was constructed beginning with pACT2 (Clontech 97/98, p. 56) as follows. The BamHI and XhoI sites in pACT2 were filled in separate steps using Klenow fragment. EcoRI was used to clone a GFP gene containing internal XhoI and BamHI restriction sites (as in pVT27, supra) into the modified pACT2. The reading frame of GFP was such that it was in frame with the DNA binding domain in pACT2. Finally, a 1.2Kb BamHI-XhoI stuffer fragment (containing 1194 coding bases of the yeast STE4 gene) was cloned into the GFP gene to yield pVT563. Perturbagen libraries are then cloned into the internal XhoI/BamHI site (or other desired internal site, as described in Ser. No. 08/965,477, supra, incorporated herein by reference). Alternatively, the perturbagen library may be cloned into positions at or near the N-terminus or C-terminus of a selected GFP. In these constructs GFP is expressed as a fusion protein with the perturbagens.

(3) Target Libraries for Yeast Two-Hybrid Assays

As described in the previous Example, genetic libraries that are particular to the therapeutic target cell of interest may be constructed. Such a target library is incorporated into a vector that is suitable for use in yeast. One such exemplary vector is pACT2, which has a selectable TRP1 marker. The vector has an ADH promoter upstream of the target cell insert to drive its expression in a constitutive manner. Alternatively, commercial libraries may be utilized. Libraries suitable for performing two hybrid selections for the purpose of identifying candidate perturbagen targets can be obtained from several sources. For example, libraries for both LexA-based and Gal4-based two hybrid selections are commercially available from a variety of companies (e.g. Clontech and Origene).

(4) Reporter Constructs for Detecting Protein-Ligand Interactions.

Validating endogenous targets as physiologically relevant candidates for therapeutic intervention involves, inter alia, the creation and characterization of reporters for detecting protein-ligand interactions. The following are exemplary, non-limiting examples of such reporter constructs.

Reporter 1—(pVT85):

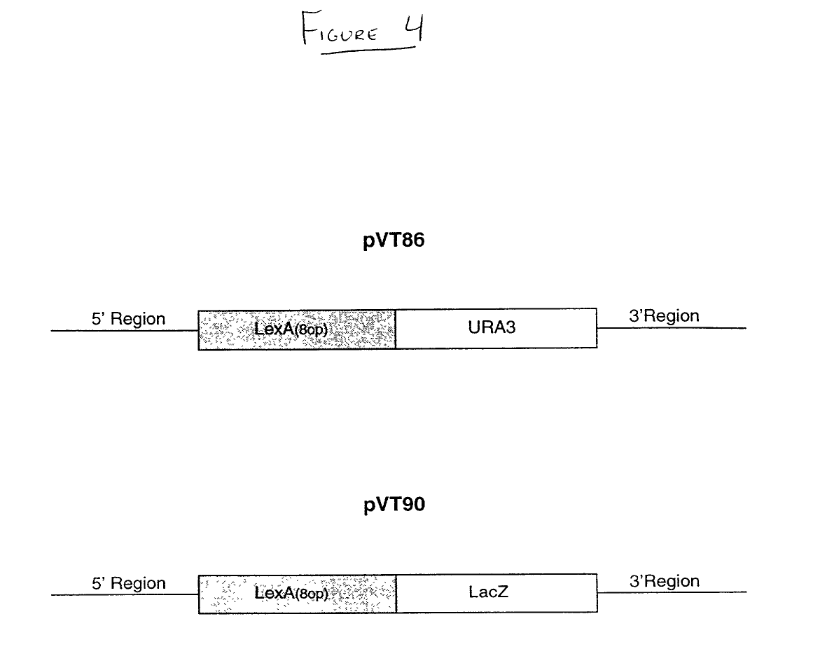

This reporter comprises the URA3 gene under the transcriptional control of the yeast Gal2 upstream activating sequence (UAS). In order to facilitate integration of this reporter into the yeast chromosome in place of the Lys2 coding region, the Gal2-Ura3 construct is flanked on the 5′ side by the 500 base pairs that lie immediately upstream of the coding region of the LYS2 gene and on the 3′ side by the 500 base pairs that lie immediately 3′ of the coding region of the LYS2 gene. FIG. 4. The entire vector is also cloned into the yeast centromere containing vector pRS413 (Sikorski, R S and Hieter, P., Genetics 122(1):19-27 (1989) and can therefore be used episomally. This reporter is intended for use with a Gal4-based two-hybrid system, e.g., Fields, S. and Song, O., Nature 340:245-246 (1989).

Reporter 2—(pVT86):

This reporter is identical to reporter #1 except that the GAL2 UAS sequences have been replaced with regulatory promoter sequences that contain eight LexA operator sequences (Ebina et al., 1983). FIG. 5. The number of LexA operator sequences in this reporter may either be increased or decreased in order to obtain the optimal level of transcriptional regulation. This reporter is intended to be used within the general confines of the LexA-based interaction trap devised by Brent and Ptashne.

Reporter 3—(pVT87):

This reporter is comprised of the yeast His3 gene under the transcriptional control of the yeast Gal1 upstream activating sequence (UAS). In order to facilitate integration of this reporter into the yeast chromosome in place of the His3 coding region the Gal1-His3 construct is flanked on the 5′ side by the 500 base pairs (bp) immediately upstream of the His3 coding region and on the 3′ side by the 500 bp immediately 3′ of the His3 coding region. FIG. 6. The entire reporter is also cloned into the yeast centromere containing vector pRS415 and can therefore be used episomally. This reporter is intended for use with a Gal4-based two-hybrid system.

Reporter 4—(pVT88):

This reporter is identical to Reporter 3 except that the His3 gene is under the transcriptional control of Gal7 UAS sequences rather than the Gal1 UAS. FIG. 7. The reporter is used with a Gal4-based two-hybrid system.

Reporter 5—(pVT89):

This reporter contains the bacterial LacZ gene under the transcriptional control of the Gal1 UAS. The entire reporter will be cloned into a yeast centromere-using vector, e.g., pRS413, and is used episomally. FIG. 8.

Reporter 6—(pVT90):

This reporter consists of the LacZ gene under the transcriptional control of eight LexA operator sequences. FIG. 9. As for Reporter 2, the number of LexA operator sequences in this reporter may either be increased or decreased in order to obtain optimal levels of transcriptional regulation. In order to facilitate integration of this reporter into the yeast chromosome in place of the Lys2 coding region, it is flanked on the 5′ side by the 500 base pairs that lie immediately upstream of the coding region of the Lys2 gene and on the 3′ side by the 500 base pairs that lie immediately 3′ of the coding region of the Lys2 gene. This reporter is used in conjunction with a LexA-based interaction trap, e.g., Golemis, E. A., et al., (1996), “Interaction trap/two hybrid system to identify interacting proteins.” Current Protocols in Molecular Biology, Ausebel et al., eds., New York, John Wiley & Sons, Chap. 20.1.1-20.1.28.

(5) Characterization of Reporter Constructs.

Following construction, all reporters are characterized in appropriate yeast strains (described herein), utilizing centromere-based vectors. Specific parameters tested are as follows.

Reporter 1:

Reporter 1 is characterized by the following steps: (a) detecting absence of growth on defined media lacking uracil and growth in the presence of 5-fluoroorotic acid (5-FOA); and (b) detecting growth in the absence of uracil and 5-FOA sensitivity in the presence of weak Gal4-transcriptional activators.

If desired, fine-tuning of this reporter in order to generate desired characteristics is accomplished by PCR-based mutagenesis of Gal2 UAS sequences combined with positive and negative selections involving uracil prototrophy and 5-FOA resistance.

Reporter 2:

Reporter 2 is characterized by the steps described above for Reporter 1, but for the exception that the weak activators in step (b) are LexA rather than Gal4 based.

If desired, fine tuning of this reporter in order to generate desired characteristics is accomplished by generating and testing different numbers of LexA operator sites using mutagenesis techniques well known to those in the art.

Reporters 3 and 4:

Reporters 3 and 4 are characterized by the following steps: (a) detecting minimal levels of growth on media lacking histidine; and (b) detecting growth on media lacking histidine in the presence of weak Gal4-transcriptional activators. One of these two reporters, and most preferably the reporter displaying more sensitive response to activation is used for the yeast strain modifications described below.

Reporters 5 and 6:

Reporters 5 and 6, which incorporate the LacZ gene, are characterized by detecting differential β-galactosidase activity in the presence of strong and weak transcriptional activators.

(6) Creation and Characterization of Exemplary Host Yeast Strains.

Construction of exemplary but non-limiting validator yeast-reporter strains is as follows.

YVT96: The starting strain was YM4271 (Liu, J. et al., 1993) MATa, ura3-52 his3-200 ade2-101 ade5 lys2-801 leu2-3, 112 trp1-901 tyr1-501 gal4Δ gal80Δ ade5::hisG. YM4271 was converted to yVT96, MATa ura3-52 his3-200 ade 2-101 ade5 lys2::GAL2-URA3 leu2-3, 112 trp1-901 tyr1-501 gal4D gal80Δ ade5::hisG by homologous recombination of Reporter 1 to the LYS2 locus. The integration is confirmed by PCR.

YVT97: The starting strain is YM4271 (Liu, J. et al., 1993) MATa, ura3-52 his3-200 ade2-101 ade5 lys2-801 leu2-3, 112 trp1-901 tyr1-501 gal4Δ gal80Δ ade5::hisG. YM4271 will be converted to yVT97, MATα ura3-52 his3::GAL1 or GAL7-HIS3 ade2-101 ade5 lys2-801 leu2-3, 112 trp1-901 tyr1-501 gal4Δ gal80Δ ade5::hisG by the steps of (a) converting from MATa to MATα via transient expression of the HO endonuclease, Methods in Enzymology Vol. 194:132-146 (1991) and (b) integrating either of Reporters 3 or 4 at the HIS3 locus via homologous recombination. The integration is confirmed by PCR.

YVT98: The starting strain is EGY48 (Estojak, J. Et al., 1995) MATα, ura3 his3 trp1 leu2::LexAop(x6)-LEU2. EGY48 is converted to strain yVT98 MATα ura3 his3 trp1 leu2::lexAop(x6)-LEU2 lys2::lexAop(8x or 2x)-LacZ by homologous recombination of Reporter 6 into the LYS2 locus.

YVT99: The starting strain is EGY48 (Estojak, J. Et al., 1995) MATα, ura3 his3 trp1 leu2::LexAop(x6)-LEU2. EGY48 is converted to strain yVT99 MATa ura3 his3 trp1 leu2::lexAop(x6)-LEU2 lys2::lexAop(8x or 2x)-URA3 by homologous recombination of Reporter 2 into the LYS2 locus and by switching the mating type from MATα to MATa via transient expression of the HO endonuclease.

YVT100: The starting strain is YM4271 (Liu, J. et al., 1993) MATa, ura3-52 his3-200 ade2-101 ade5 lys2-801 leu2-3, 112 trp1-901 tyr1-501 gal4Δ gal80Δ ade5::hisG. YM4271 was converted to yVT100, MATa ura3-52 his3-200 ade2-101 ade5 lys2::lexAop(8x or 2x)-URA3 leu2-3, 112 trp1-901 tyr-501 gal4Δ gal80Δ ade5::hisG by homologous recombination of Reporter 2 to the LYS2 locus. The integration is confirmed by PCR.

### EXAMPLE 3

### Identifying Physiologically Relevant Targets in a Melamona Cell Line

The invention can be applied to find targets of perturbagens that have been isolated using selections/screens in mammalian cells. As an example, perturbagen libraries are introduced by retroviral gene transfer into HS294T melanoma cells that contain a regulated p16 gene. The induction of this gene leads to cell cycle arrest and ultimately death caused by p16 overexpression. Cells that escape from p16-mediated arrest and death are recovered following this first phenotypic assay. The resident perturbagens are isolated by PCR amplification using primers specific to the perturbagen flanking DNA sequences.

Next, a first protein interaction assay isolates the endogenous cellular components that bound to the first set of perturbagens. The perturbagen sequences are cloned so as to produce BD Gal4 or LexA fusions in a yeast expression vector and introduced into haploid yeast. The yeast strain used for the initial two hybrid selection in the case of the Gal4 based system is, e.g., pVT97. Alternatively, the perturbagens are cloned into a LexA based system, and yeast strain yVT98 is used. This first assay consists of an initial selection on either defined media lacking histidine (Gal4 system) or leucine (LexA system), followed by an optional secondary screen for prey-bait interaction that monitors resultant expression of the LacZ reporter. Plasmid DNA encoding candidate targets can be recovered individually from surviving yeast by standard procedures.

An optional step to remove some artifactual false positives prior to recovery of the DNA is performed in the following manner. Individual survivors of the first round can be pooled and induced to lose the perturbagen containing plasmid through growth in non-selective media and/or use of a negative selection. Yeast harboring only the candidate perturbagen encoding plasmids will then be mated to strains yVT96 (Gal4) or yVT99 or yVT100 (LexA) that harbor “false baits” such as the lamin protein. Selection for diploids can then be carried out in the presence of 5-FOA. In this manner only diploids are enriched for cells that will grow and form colonies. DNA from the therapeutic target cell line used in the phenotypic assay is then recovered by standard methods.

Next, another protein interaction assay is performed. The second round of two hybrid selections occurs between the putative therapeutic targets (endogenous molecules that bound to the first set of perturbagens) and a second, independent perturbagen library—e.g., a random-primed library of, e.g., human fetal brain mRNA, expressed as fusions with the Gal4 AD or a peptide library in GFP fused to the Gal4 BD. These selections involve a mating between yeast strains harboring one or more of the candidate targets and yeast strains harboring the appropriate perturbagen probe libraries. Strains used are yVY96 and 97 in the case of the Gal4 system and yVT98 and either vVT99 or yVT100 in the case of the LexA system. Candidate targets may be subcloned to the binding domain side prior to these selections (with self-activators removed as described above). These selections will be carried out as in the first round, except that false positives will not be depleted following the selection.

Optionally, the set of primary phenotypic probe sequences is tested against a random-primed library of, e.g., human fetal brain mRNA, expressed as fusions with the Gal4 AD. Mated diploid yeast that contain both a clone from the perturbagen set and a clone from the prey set are plated on media lacking URA and the URA3+ phenotype is selected. Surviving cells are isolated and the candidate targets are recovered from the prey library as Gal4 AD fusions. These candidate target sequences are reintroduced into yeast and tested against a depleted peptide library as described in the previous example. The URA3+ phenotype signifying a physical interaction between a particular peptide fusion and a candidate target fusion is selected and the peptide fusion sequences are recovered.

Next, a second phenotypic assay is performed as follows. The recovered peptides are recloned into a mammalian expression vector, e.g., a retroviral vector. These sequences are introduced once again into HS294T melanoma cells engineered with p16 and the cells are subjected to selection wherein escape from p16-mediated arrest and death is required. The cells that pass this test and form colonies are recovered and their resident perturbagen-encoding sequences isolated by PCR. These sequences are tested against the set of candidate targets in the same manner as described above, involving a selection on media lacking either histidine (Gal4) or leucine (LexA) and a secondary screen that monitors expression of the LacZ gene. A candidate target that binds to one of the confirmatory phenotypic probes is thus identified as a validated, physiologically relevant target.

### EXAMPLE 4

### Optional Steps for Improving the Efficiency of a Yeast Two-Hybrid Protein Interaction Assay

In some cases it may be desirable to switch the candidate targets from the activation domain side to the binding domain side between the first and second rounds of two-hybrid selections. This can be accomplished in a number of ways that use standard practices of molecular biology including, but not limited to, PCR, subcloning and gap repair.

Also in some cases it may be desirable to remove self-activating sequences from two-hybrid libraries prior to a two hybrid selection. This is most important in the case of protein or peptide fusions with the Gal4 and LexA DNA binding domains as a large percentage of random sequences can activate transcription. To remove self-activating sequences from DNA binding domain fusions with candidate phenotypic probes (perturbagens), an initial negative selection is performed using strains yVT96 (Gal4) and either yVT99 or yVT100 (LexA). Yeast harboring DNA-binding domain libraries will be grown in the presence of 5-FOA thereby eliminating yeast that harbor cells that express the Ura3 gene. Yeast that survive this selection and therefore collectively contain libraries depleted of self-activating sequences will be pooled and frozen. Aliquots of these yeast can then be thawed and used in the second round of selection. Similar negative selections can also be performed on binding domain-cDNA libraries in order to facilitate two hybrid selections involving candidate targets that self activate transcription.

### EXAMPLE 5

Using Biochemical Methods to Detect Validated Protein-ligand Interactions.

As an alternative to yeast two-hybrid protein interaction assays, it is possible to use affinity purification to identify endogenous proteins from therapeutic target cells that bind to perturbagen. The first step involves use of at least one perturbagen as an affinity reagent to select from a cell extract proteins that bind the perturbagen(s). This is performed with individual perturbagens, or alternatively, en masse with a collection of perturbagens. The perturbagens preferably have attached to them a label that permits the use of a generic binding matrix to attach them to a solid support. Examples include the FLAG epitope, HisTag, maltose-binding domain, glutathione-S-transferase, and others.

After incubation with the cell extract under conditions of salt, pH, etc. appropriate for binding and affinity purification. In many cases, conditions that reproduce physiological pH and salt levels in the cell are appropriate. In other cases, conditions that permit binding between the label or tag and an affinity matrix are demanded (e.g., conditions suitable for interaction between glutatione-S-transferase and its ligand, glutathione. These conditions can be gleaned from standard suppliers' instructions, or from standard molecular biology protocols. The perturbagen(s) and their attached cellular proteins are separated from the bulk of unbound cellular proteins by a series of routine washing steps designed to remove non-specifically bound proteins. The enriched complexes of bound proteins and perturbagen(s) are collected for analysis.

Next, one analyzes the protein(s) bound to a single or set of perturbagens. One particularly attractive method of doing so is to use recently-developed mass spectrometric methods. Mass spec instruments are commercially available and can be used in a variety of contexts to analyze macromolecules including proteins. In one version, the sample of perturbagen-bound proteins is first proteolyzed with a specific protease or collection of proteases, fractionated on a HPLC (high pressure liquid chromatography) column, and subjected to MALDI mass spec. From the peaks that are detected, charge/mass ratios are measured and amino acid composition of individual peptide fragments are inferred. The amino acid compositions can be compared against predicted fragments from a protein or translated DNA database. If matches are found, perturbagen-binding partners can be identified based on the match, typically with a high degree of confidence. The sum of all the database “hits” in principle defines the family of candidate perturbagen-binding proteins in the original sample.

The next step in the process requires identification of peptides or protein fragments that physically interact with individual members of the family of perturbagen-binding proteins. One biochemical strategy for isolation of such agents involves the use of expressed, purified protein using phage display. Full length cDNA encoding the above-identified binding partners can be constructed or obtained from commercial organizations. These clones can either be transferred into suitable expression constructs or used directly to produce in, e.g., E. coli a substantial quantity of the given protein. The protein can be purified by a variety of methods known in the art and used as the basis for phage display experiments. In these experiments, the purified protein is typically attached to a solid support and serves to select from a library of peptides displayed on the surface of phage a set of secondary candidate perturbagens.

Finally, one identifies the physiologically relevant binding partners. For example, the set of DNA fragments encoding these candidate confirmatory perturbagens can be cloned into a mammalian expression vector, e.g., and the entire population can be introduced into the assay originally used to isolate the primary perturbagens. Those secondary perturbagens that are recovered from the assay (i.e., that have physiological effects similar to the primary perturbagens) are derived from specific candidate targets; that is, they bind to specific candidate targets identified as above. The candidate targets that bind both primary and secondary perturbagens as judged by biochemical experiments are the physiologically relevant binding partners, i.e., the perturbagen targets in vivo.

### EXAMPLE 6

### Validation of an Endogenous Target in Yeast

As an example of the application of the invention to screening in yeast, a series of experiments led to identification of perturbagens that confer resistance to growth arrest caused by pheromone (Caponigro et al., 1998). Some of the binding partners of these perturbagens were defined by standard yeast two-hybrid experiments against known components of the pheromone response pathway. However, other perturbagen targets were not identified.

To find the unknown targets, the methods of the invention are applied in yeast. First, the perturbagens in question are expressed as fusions with the Gal4 BD in yeast cells. These fusions are the “bait” and are tested for interaction with members of a prey library consisting of randomly sheared yeast genomic DNA (gDNA) cloned to encode fusions with the Gal4 AD on a yeast expression plasmid. The bait and prey libraries are examined together in yeast cells by mating the haploid library-containing yeast to form diploids that contain one (sometime more than one) member of the bait library and prey library. Selection for URA3+ defines a subset of prey sequences that interact physically with bait sequences. These are collected using PCR amplification or plasmid isolation.

The AD fusion sublibrary can be used directly against a library of C-terminal peptides (15 amino acids) displayed on a GFP scaffold that is fused to the Gal4 BD. This prey library has been depleted of members that activate in the absence of a second physical interaction by negative selection against the URA3+ phenotype. The depleted library is mated to yeast cells that contain the bait constructs (introduced by yeast transformation into haploid yeast) and URA3+ diploids are recovered. From these surviving cells, the peptides are isolated and recloned into a galactose-regulated expression vector that contains GFP, capable of expressing peptides fused to the GFP C-terminus.

The sublibrary of GFP-peptide fusions is reintroduced into yeast cells and yeast are identified that grow in the presence of pheromone and galactose. These yeast are further tested to ensure that their escape is galactose-dependent. Those that express peptides that confer resistance to pheromone are collected and used in a second focused two-hybrid assay to identify binding partners from the original set of candidate targets. The candidate targets from the original prey library which bind to any member of the second set of perturbagens are considered to be valid in vivo targets having physiological relevance that may be potentially used in, for example, development of anti-fungal agents, or alternatively may be extrapolated to human physiological pathways.

### EXAMPLE 7

### Validation of an Endogenous Target in Virally Infected Cells

Perturbagens can be used to identify points of vulnerability in the pathways involved in viral infection. These points of vulnerability may include viral proteins or cellular proteins required by the virus for productive infection.

As an example, adenovirus infects humans producing in some cases cold-like symptoms. To find adenovirus targets for antiinfective drugs, adenovirus was engineered to contain the GFP gene regulated by the CMV promoter (Adeno-GFP, Cat. No. AES0515, Quantum Biotechnologies, Montreal, PQ, Canada) Cells productively infected by this virus fluoresce bright green, and thus can be readily visualized or sorted by standard methods.

Epstein-Barr viral vectors containing the putative perturbagen encoding sequences were constructed as follows: GFP was mutated at codon 66 (Y66F) in order to eradicate fluorescence (“dead” GFP). Perturbagen-encoding sequences were then inserted into the dead GFP scaffold at the C-terminus. Two perturbagen libraries were constructed: the first library utilized synthetic peptides, the second utilized cDNA derived from human placenta polyA+ mRNA.

The perturbagen constructs were transfected into human 293 cells using lipofection and allowed to express the perturbagen/dead GFP fusions for two days. These perturbagen-containing cells were then infected at a MOI of 10 with the recombinant adenovirus expressing fluorescent (“live”) GFP. In order to enrich the population for cells that are not productively infected with adenovirus, the cell population was trypsinized 36 hours after infection. Cells that do not subsequently re-adhere were removed by washing, when the cells were harvested at 48 hours. The cells were then sorted by flow cytometry. Those cells that were dim (i.e., exhibiting low fluorescence) were recovered by flow sorter and their resident perturbagen-encoding sequences are recovered by PCR.

After two cycles of reintroduction and infection, the perturbagens that confer resistance to adenovirus infection are identified and their encoding sequences are cloned into a BD vector. Validated, physiologically relevant targets are identified by pursuing the same steps as described in the previous example.

The above examples are provided to illustrate the invention but not to limit its scope. Other variants of the invention will be readily apparent to one of ordinary skill in the art and encompassed by the appended claims. All publications, patents, and patent applications cited herein are hereby incorporated by reference.

What is claimed is:

1. A method for reducing false positives from an assay that identifies protein interactions, comprising the steps of:
a) selecting a pool of putative target molecules that interact with a first phenotypic probe in a first protein interaction assay;
b) selecting a pool of second independent probes that interact with the pool of putative target molecules in a second protein interaction assay;
c) selecting from the pool of second independent probes at least one confirmatory phenotypic probe that is capable of altering a phenotype of interest in a phenotypic assay host cell; and
d) identifying members of the pool of putative target molecules that interact with both the first phenotypic probe and the confirmatory phenotypic probe.

2. A method for identifying a physiologically relevant target molecule that correlates to a phenotype of interest, comprising the steps of:
(a) determining a first protein-ligand interaction between a pool of target molecules and a first physiologically relevant probe that confers a first phenotype of interest on a host cell;
(b) determining a second protein-ligand interaction between the pool of target molecules and a second independent physiologically relevant probe that confers a second phenotype of interest on a host cell; and
(c) isolating any target molecule that interacts with both of the first and second probes.

3. The method of claim 2, wherein the first and second protein-ligand interactions are determined by performing a first and second yeast two-hybrid assay.

4. The method of claim 3, wherein the first yeast two-hybrid assay utilizes the pool of target molecules as prey and the second yeast two-hybrid assay uses the pool of target molecules as bait.

5. The method of claim 2, wherein said first and said second phenotypes of interest are the same cellular characteristic.

6. The method of claim 2, wherein said first and said second phenotypes of interest are related cellular characteristics.

7. A method for identifying a physiologically relevant target that correlates to a phenotype of interest, comprising the steps of:
(a) exposing a primary phenotypic probe to a candidate target library;
(b) identifying a pool of putative target molecules that interact with the primary phenotypic probe;
(c) exposing the pool of putative target molecules to a library of candidate secondary probes;
(d) identifying a sublibrary within said library of candidate secondary probes that interacts with the pool of putative target molecules;
(e) selecting from said sublibrary a confirmatory probe that alters a phenotype of interest in a host cell; and
(f) identifying members of the pool of putative target molecules that interact with the confirmatory probe.

8. The method of claim 7, wherein the pool of putative target molecules are perturbagen binding partners.

9. The method of claim 8, wherein said perturbagen binding partners are polypeptides.

10. The method of claim 7, wherein the candidate target library is an expression library of recombinant polypeptides.

11. The method of claim 10, wherein the expression library is encoded by genomic DNA.

12. The method of claim 10, wherein the expression library is encoded by cDNA.

13. The method of claim 7, wherein the primary and secondary phenotypic probes are perturbagens.

14. The method of claim 13, further comprising the step of fusing at least one of the perturbagens to a stabilizing polypeptide.

15. The method of claim 14, wherein the stabilizing polypeptide is GFP.

16. The method of claim 7, wherein the steps of exposing the primary and secondary probes to the pool of target molecules are performed by a first and a second yeast two-hybrid assay.

17. The method of claim 16, wherein the first yeast two-hybrid assay utilizes members of the candidate target library as prey and the second yeast two-hybrid assay uses the pool of target molecules as bait.

18. The method of claim 16, further comprising the step of eliminating bait sequences that self-activate.

19. The method of claim 16, wherein the yeast two-hybrid system utilizes a GAL4-based reporter system.

20. The method of claim 16, wherein the yeast two-hybrid system utilizes LexA-based reporter system.

21. The method of claim 19, wherein the yeast two-hybrid system utilizes a reporter vector selected from the group consisting of pVT85, pVT87, pVT88 and pVT89.

22. The method of claim 20, wherein the yeast two-hybrid system utilizes a reporter vector selected from the group consisting of pVT86 and pVT90.

23. The method of claim 19, wherein the yeast two-hybrid system utilizes a yeast strain selected from the group consisting of yVT96 and yVT97.

24. The method of claim 20, wherein the yeast two-hybrid system utilizes a yeast strain selected from the group consisting of yVT98 and yVT99.
